(19) United States
(12) Patent Application Publication
(10) Pub. No.: US 2015/0148574 A1
(43) Pub. Date: May 28, 2015

(54) PROCESS FOR THE PREPARATION OF AN OLEFIN PRODUCT

(71) Applicants: SHELL INTERNATIONALE RESEARCH MAATSCHAPPIJ B.V., The Hague (NL); SHELL OIL COMPANY, Houston, TX (US)

(72) Inventors: Leslie Andrew Chewter, Amsterdam (NL); Timothy Michael Nisbet, Amsterdam (NL); Sivakumar Sadasivan Vijayakumari, Amsterdam (NL); Jeroen Van Westrenen, Amsterdam (NL)

(21) Appl. No.: 14/368,516

(22) PCT Filed: Dec. 20, 2012
(86) PCT No.: PCT/EP2012/076436

§ 371 Date: Jun. 25, 2014

(30) Foreign Application Priority Data
Dec. 27, 2011 (EP) 11195819.5

(51) Int. Cl. C07C 1/20; C07C 4/06

(52) U.S. Cl. CPC C07C 1/20; C07C 4/06; C07C 2529/40
USPC 585/316; 585/640

(57) ABSTRACT

A process for the preparation of an olefin product, which process comprises the steps of: a) converting an oxygenate feedstock in an oxygenate-to-olefins conversion system, comprising a reaction zone in which an oxygenate feedstock is contacted with an oxygenate conversion catalyst under oxygenate conversion conditions, to obtain a conversion effluent comprising olefins and paraffins; b) separating at least a portion of the paraffins from the conversion effluent to form a paraffin stream; and c) recycling at least a portion of the paraffin stream to step a).

### FIELD OF THE INVENTION

The invention relates to a process for preparing lower olefins.

### BACKGROUND

Oxygenate-to-olefin processes are well described in the art. Typically, oxygenate-to-olefin processes are used to produce predominantly ethylene and propylene. An example of such an oxygenate-to-olefin process is described in US Patent Application Publication No. 2011/112344, which is herein incorporated by reference. The publication describes a process for the preparation of an olefin product comprising ethylene and/or propylene, comprising a step of converting an oxygenate feedstock in an oxygenate-to-olefins conversion system, comprising a reaction zone in which an oxygenate feedstock is contacted with an oxygenate conversion catalyst under oxygenate conversion conditions, to obtain a conversion effluent comprising ethylene and/or propylene.

The publication further describes possible integration with a cracker. The publication also describes partially hydrogenating a C₄ portion of the conversion effluent and/or cracker effluent and recycling at least part of the at least partially hydrogenated C₄ as recycle feedstock to the cracker or oxygenate-to-olefins conversion system.

The oxygenate-to-olefins process typically produces additional products, for example, C₄+ olefins and C₄+ paraffins. The olefins can be recycled to the oxygenate-to-olefins process, but the paraffins are typically purged from the system via a bleed line. It would be advantageous to develop a way to convert the C₄+ paraffins to valuable olefinic containing products.

### SUMMARY OF THE INVENTION

The invention provides a process for the preparation of an olefin product which process comprises the steps of: a) converting an oxygenate feedstock in an oxygenate-to-olefins conversion system, comprising a reaction zone in which an oxygenate feedstock is contacted with an oxygenate conversion catalyst under oxygenate conversion conditions, to obtain a conversion effluent comprising olefins and paraffins; b) separating at least a portion of the paraffins from the conversion effluent to form a paraffin stream; and c) recycling at least a portion of the paraffin stream to step a).

### BRIEF DESCRIPTION OF THE DRAWINGS

FIG. 1 depicts an embodiment of a process flow scheme in accordance with the invention.

### DETAILED DESCRIPTION

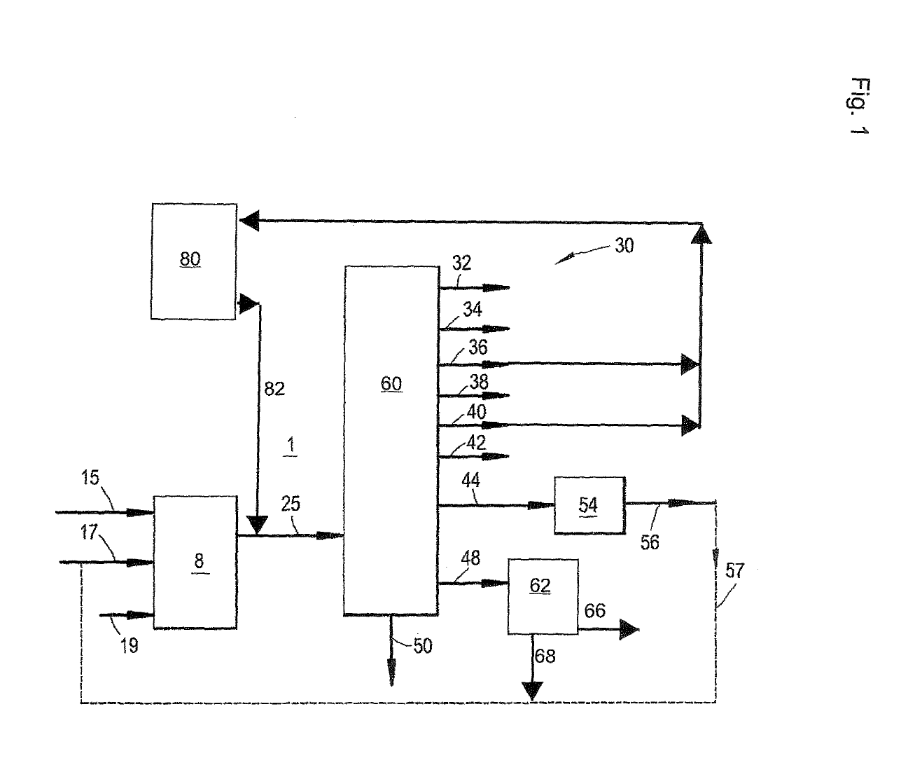

Reference is made to FIG. 1, showing an embodiment of a process flow scheme for an oxygenate-to-olefins conversion process.

The process comprises an oxygenate-to-olefins (OTO) conversion system 8 and a work-up section 60. An oxygenate feedstock is fed via line 15 to the OTO conversion system 8, for example, comprising methanol and/or dimethylether. Optionally, a hydrocarbon stream and/or a diluent are fed to the OTO conversion system via lines 17 or 19, respectively.

In principle every known OTO conversion system and process can be used in conjunction with the present invention, including processes known as Methanol-to-Olefins (MtO) and Methanol to Propylene (MtP). The OTO conversion system and process can for example be as disclosed in US 2005/0038304, incorporated herein by reference; as disclosed in US 2010/206771, incorporated herein by reference; or as disclosed in US 2006/020155 incorporated herein by reference. Other particularly suitable OTO conversion processes and systems with specific advantages are disclosed in US 2009/187058, US 2010/298619, US 2010/268009, US 2010/268007, US 2010/261943, and US 2011/160509, all of which are herein incorporated by reference.

In one embodiment, molecular sieve catalysts are used to convert oxygenate compounds to light olefins. Silicoaluminophosphate (SAPO) molecular sieve catalyst may be used that are selective to the formation of ethylene and propylene. Preferred SAPO catalysts are SAPO-17, SAPO-18, SAPO-34, SAPO-35, SAPO-44, the substituted forms thereof and mixtures thereof. The oxygenate feedstock may comprise one or more aliphatic containing compounds, including alcohols, amines, carbonyl compounds, for example, aldehydes, ketones and carboxylic acids, ethers, halides, mercaptans, sulfides, and the like and mixtures thereof. Examples of suitable feedstocks include methanol, ethanol, methyl mercaptan, ethyl mercaptan, methyl sulfide, methyl amine, di-methyl ether, di-ethyl ether, methyl ethyl ether, methyl chloride, ethyl chloride, dimethyl ketone, formaldehyde, acetaldehyde and various acids such as acetic acid.

In one embodiment, the oxygenate feedstock comprises one or more alcohols having from 1 to 4 carbon atoms and most preferably methanol. The oxygenate feedstock is contacted with a molecular sieve catalyst and is converted to light olefins, preferably ethylene and propylene.

Preferably, the OTO conversion system is arranged to receive an olefin stream and/or a paraffin stream, and is able to at least partially convert these streams to different olefins and/or paraffins. The olefins and paraffins can be contacted with the oxygenate conversion catalyst in the OTO reaction zone as described in US 2009/187058, US 2010/298619 and US 2010/268009.

In another embodiment, the OTO conversion system comprises an olefin cracking zone downstream from the OTO reaction zone and is arranged to crack C₄₊ olefins and/or aromatics produced in the OTO reaction zone, as described in U.S. Pat. No. 6,809,227 and US 2004/0102667. In this embodiment, at least a portion of the olefins produced in the OTO conversion are fed to the olefin cracking zone.

In one embodiment, an olefinic co-feed is fed to the oxygenate-to-olefins conversion system. An olefinic co-feed is a feed containing one or more olefins or a mixture of olefins. The olefinic co-feed may also comprise other hydrocarbon compounds, for example, paraffinic compounds, alkylaromatic compounds, aromatic compounds or mixtures thereof. The olefinic co-feed preferably comprises more than 25 wt % olefins, more preferably more than 50 wt %, still more preferably more than 80 wt % and most preferably in the range of from 95 to 100 wt % olefins. A preferred olefinic co-feed consists essentially of olefins. Non-olefinic compounds in the olefinic co-feed are preferably paraffinic compounds.

The olefins in the olefinic co-feed are preferably mono-olefins. Further, the olefins can be linear, branched or cyclic, but they are preferably linear or branched. The olefins may have from 2 to 12 carbon atoms, preferably 3 to 10 carbon atoms and more preferably from 4 to 8 carbon atoms.

In one embodiment, a paraffinic co-feed is fed to the oxygenate-to-olefins conversion system. A paraffinic co-feed is a feed containing one or more paraffin compounds or a mixture of paraffinic compounds. The paraffinic co-feed may also comprise other hydrocarbon compounds, for example, aromatic compounds, olefinic compounds or mixtures thereof. The paraffinic co-feed preferably comprises more than 25 wt % paraffins, more preferably more than 50 wt %, still more preferably more than 80 wt % and most preferably in the range of from 95 to 100 wt % paraffins. A preferred paraffinic co-feed consists essentially of paraffins. Non-paraffinic compounds in the paraffinic co-feed are preferably olefinic compounds. A preferred paraffinic co-feed comprises pentane.

The paraffins can be fed to the OTO conversion system and/or to a cracking unit. The paraffins may be fed alone or with olefins to either of these units. In one embodiment, the paraffins and C₄ olefin stream can be fed to the OTO conversion system while a C₅ and C₆ olefin stream is fed to an olefin cracking unit. In another embodiment, the C₅ paraffins can be co-fed to an olefin cracking unit. Any remaining paraffins and/or olefins produced in the cracking unit can optionally be fed to the OTO conversion system. In another embodiment, the C₅ paraffins can be co-fed to an olefin cracking unit while the C₄ olefins are fed to the OTO conversion system.

Both the OTO process and the optional catalytic olefin cracking process may be operated in a fluidized bed or moving bed, e.g. a fast fluidized bed or a riser reactor system, and also in a fixed bed reactor or a tubular reactor. A fluidized bed or moving bed, e.g. a fast fluidized bed or a riser reactor system are preferred.

Catalysts suitable for converting the oxygenate feedstock preferably include molecular sieve-comprising catalyst compositions. Such molecular sieve-comprising catalyst compositions typically also include binder materials, matrix material and optionally fillers. Suitable matrix materials include clays, such as kaolin. Suitable binder materials include silica, alumina, silica-alumina, titania and zirconia, wherein silica is preferred due to its low acidity.

Molecular sieves preferably have a molecular framework of one, preferably two or more corner-sharing [TO₄] tetrahedral units, more preferably, two or more [SiO₄], [AlO₄] and/or [PO₄] tetrahedral units. These silicon, aluminum and/or phosphorus based molecular sieves and metal containing silicon, aluminum and/or phosphorus based molecular sieves have been described in detail in numerous publications including for example, U.S. Pat. No. 4,567,029. In a preferred embodiment, the molecular sieves have 8-, 10- or 12-ring structures and an average pore size in the range of from about 3 Å to 15 Å.

Suitable oxygenate-to-olefins conversion catalysts are aluminosilicate-comprising catalysts, in particular a zeolite-comprising catalyst. Suitable catalysts include those containing a zeolite of the ZSM group, in particular of the MFI type, such as ZSM-5, the MTT type, such as ZSM-23, the TON type, such as ZSM-22, the MEL type, such as ZSM-11, the FER type. Other suitable zeolites are for example zeolites of the STF-type, such as SSZ-35, the SFF type, such as SSZ-44 and the EU-2 type, such as ZSM-48.

Aluminosilicate-comprising catalysts, and in particular zeolite-comprising catalysts, have the additional advantage that in addition to the conversion of methanol or ethanol, these catalysts also induce the conversion of olefins to ethylene and/or propylene. Furthermore, these aluminosilicate-comprising catalysts, and in particular zeolite-comprising catalysts, are particularly suitable for use as the catalyst in a catalytic olefin cracking zone. Particular preferred catalyst for this reaction, i.e. converting part of the olefins as well as converting paraffins to an olefinic product, are catalysts comprising at least one zeolite selected from MFI, MEL, TON and MTT type zeolites, more preferably at least one of ZSM-5, ZSM-11, ZSM-22 and ZSM-23 zeolites.

Preferred catalysts, for both the OTO reaction as well as an optional catalytic olefin cracking reaction, comprise a more-dimensional zeolite, in particular of the MFI type, more in particular ZSM-5, or of the MEL type, such as zeolite ZSM-11. Such zeolites are particularly suitable for converting paraffins and olefins, including iso-olefins, to ethylene and/or propylene. The zeolite having more-dimensional channels has intersecting channels in at least two directions. So, for example, the channel structure is formed of substantially parallel channels in a first direction, and substantially parallel channels in a second direction, wherein channels in the first and second directions intersect. Intersections with a further channel type are also possible. Preferably the channels in at least one of the directions are 10-membered ring channels. A preferred MFI-type zeolite has a Silica-to-Alumina ratio SAR of at least 60, preferably at least 80.

Particular catalysts, for both the OTO reaction as well as an optional olefin cracking reaction, include catalysts comprising one or more zeolite having one-dimensional 10-membered ring channels, i.e. one-dimensional 10-membered ring channels, which are not intersected by other channels. Preferred examples are zeolites of the MTT and/or TON type. Preferably, the catalyst comprises at least 40 wt %, preferably at least 50 wt % of such zeolites based on total zeolites in the catalyst.

In a particularly preferred embodiment the catalyst, for both the OTO reaction as well as an optional catalytic olefin cracking reaction, comprises in addition to one or more one-dimensional zeolites having 10-membered ring channels, such as of the MTT and/or TON type, a more-dimensional zeolite, in particular of the MFI type, more in particular ZSM-5, or of the MEL type, such as zeolite ZSM-11.

The catalyst, for both the OTO reaction as well as an optional catalytic olefin cracking reaction, may comprise phosphorus as such or in a compound, i.e. phosphorus other than any phosphorus included in the framework of the molecular sieve. It is preferred that a MEL or MFI-type zeolites comprising catalyst additionally comprises phosphorus. The phosphorus may be introduced by pre-treating the MEL or MFI-type zeolites prior to formulating the catalyst and/or by post-treating the formulated catalyst comprising the MEL or MFI-type zeolites. Preferably, the catalyst comprising MEL or MFI-type zeolites comprises phosphorus as such or in a compound in an elemental amount of from 0.05-10 wt % based on the weight of the formulated catalyst. A particularly preferred catalyst comprises MEL or MFI-type zeolites having SAR of in the range of from 60 to 150, more preferably of from 80 to 100, and phosphorus, wherein the phosphorus has preferably been introduced by post-treatment of the formulated catalyst. An even more particularly preferred catalyst comprises ZSM-5 having SAR of in the range of from 60 to 150, more preferably of from 80 to 100, and phosphorus, wherein the phosphorus has preferably been introduced by post-treatment of the formulated catalyst.

It is preferred that molecular sieves in the hydrogen form are used in the oxygenate conversion catalyst in step (g), e.g., HZSM-22, HZSM-23, and HZSM-48, HZSM-5. Preferably at least 50 wt %, more preferably at least 90 wt %, still more preferably at least 95 wt % and most preferably 100 wt % of the total amount of molecular sieve used is in the hydrogen form. It is well known in the art how to produce such molecular sieves in the hydrogen form.

Typically the catalyst deactivates in the course of the process, primarily due to deposition of coke on the catalyst. Conventional catalyst regeneration techniques can be employed to remove the coke. It is not necessary to remove all the coke from the catalyst as it is believed that a small amount of residual coke may enhance the catalyst performance and additionally, it is believed that complete removal of the coke may also lead to degradation of the molecular sieve. This applies to the catalyst for both the OTO reaction as well as an optional catalytic olefin cracking reaction.

In one embodiment different catalysts are used in the OTO conversion system and the olefin cracking unit. A SAPO molecular sieve catalyst is employed in the OTO conversion system and a zeolitic catalyst is employed in the olefin cracking unit. In this embodiment, the C₅ paraffins and C₄ olefins are passed through the olefin cracking unit in the absence of methanol.

The catalyst particles used in the process of the present invention can have any shape known to the skilled person to be suitable for this purpose. The catalyst can be present in the form of spray dried catalyst particles, spheres, tablets, rings, or extrudates. Extruded catalysts can be applied in various shapes, such as, cylinders and trilobes. If desired, spent oxygenate conversion catalyst can be regenerated and recycled to the process of the invention. Spray-dried particles that are suitable for use in a fluidized bed or riser reactor system are preferred. Spherical particles are normally obtained by spray drying. Preferably the average particle size is in the range of 1-200 μm, preferably 50-100 μm.

Suitable OTO processes will be further described in detail below. In the OTO conversion system 8, the oxygenate feedstock, a paraffin stream and optionally an olefin co-feed (both of which can be partly or fully a recycle stream) are contacted with an oxygenate conversion catalyst under oxygenate conversion conditions, to obtain a conversion effluent comprising olefins in line 25. The paraffins and olefins may be fed to the OTO conversion system together or separately. An optional diluent stream may comprise water, steam, inert gases such as nitrogen and methane.

The reaction conditions of the oxygenate conversion include a reaction temperature of 600 to 660° C., preferably from 600 to 640° C., more preferably 620 to 640° C.; and a pressure from 0.1 kPa (1 mbar) to 5 MPa (50 bar), preferably from 100 kPa (1 bar) to 1.5 MPa (15 bar).

Although applicants do not wish to be bound by this theory, it is believed that the paraffins fed to the OTO reactor crack to form lower olefins and smaller paraffins, for example, C₅ paraffins may crack to form a C₄ olefin and methane, ethane and propylene or C₅ olefins and hydrogen. In the presence of methanol, the C4 olefin reacts with methanol to form C5 and higher olefins which are likely then cracked to an ethylene and a propylene molecule.

Additionally, ethane is formed in this process and this may be fed to an optional steam cracking unit or furnace. The products from this cracking unit or furnace are preferably combined with the conversion effluent from the OTO process and fed to the workup section.

Effluents from the OTO conversion system need to be worked up in order to separate and purify various components as desired, and in particular to separate paraffin components and one or more lower olefin product streams. FIG. 1 shows a work-up section 60 which receives and processes at least part of the conversion effluent.

Typically, the effluent is quenched in a quench unit with a quench medium such as water to cool the process gas before feeding it to a compressor. This allows for a smaller compressor and lower power consumption due to reduced gas volume. Any liquid hydrocarbons after the quench are phase separated from liquid water and separately recovered. The water or steam recovered from the quench unit can be partially recycled as diluent to the OTO conversion system via line 19. The water may be treated or purified, for example, to remove catalyst fines or to maintain the pH around neutral.

The vapor components after the quench are typically sent to a compression section that can comprise multiple compression steps, subjected to a caustic wash treatment, dried and sent to a separation including a cold section, to obtain separate streams of the main components. Additional compression steps may be carried out during, or after any of the above mentioned washing and drying steps. FIG. 1 shows hydrogen stream 32, light ends stream 34 typically comprising hydrogen, methane and/or carbon monoxide, ethane stream 36, ethylene stream 38, propane stream 40, propylene stream 42, a C₄ stream 44, a C₅₊ stream 48 and a water effluent 50. There can also be a separate outlet for heavy (liquid) hydrocarbons. As known to one of ordinary skill in the art, the work-up section may be designed to provide different purities of each stream, and some of the streams will be produced from the work-up section as combined streams, i.e., C₄, C₅ and C₆ components can be combined. Additional reaction, treatment and/or purification steps may be carried out on any of these streams. For example, methane, carbon monoxide and hydrogen may be fed to a methanator to produce methane.

It is advantageous to recycle at least part of the various streams to the OTO conversion system 8. This invention provides an increased production of lower olefins by recycling the paraffins along with optional recycling of the C₄ and/or other olefin streams.

Some changes may be necessary to allow the system to handle the recycle of a portion of or the entire paraffin stream. For example, it is beneficial to keep the partial pressures of ethylene and propylene low in the OTO conversion system to prevent benzene and/or toluene from alkylating with the ethylene and propylene. Further, it is preferred to maintain a temperature in the range of from 600° C. to 660° C., preferably in the range of from 600° C. to 640° C. In a preferred embodiment the average temperature is between 620 and 640° C.

In the workup section, one of ordinary skill in the art will be able to apply any of the suitable means to separate the paraffins from the olefins. One example of a suitable method is extractive distillation.

In one embodiment, the C5 olefins and paraffins are not separated from each other due to the difficulty of separating these close-boiling components. The combined C5 stream is cracked to lower range carbon numbers and the propane is separated from propylene and the ethane from ethylene. A portion of the paraffins may thus be removed before recycling to the OTO conversion system, if desired. The ethane produced may be cracked in an ethane cracker to produce additional ethylene.

FIG. 1 shows a C₅+ olefin stream 66 being produced from the workup section. In one embodiment this is recycled to the OTO conversion system.

FIG. 1 shows paraffin stream 68, and a portion or this entire stream may be recycled to the OTO conversion system 8. The paraffin stream could be fed with the C₄ recycle via lines 57 and 17. One of ordinary skill in the art will recognize that the work-up section could be operated such that the paraffin and C₄₊ streams are not separated, but fed together to the OTO conversion system.

In one embodiment, the C₄ stream may be separated and the C₅+ olefin stream and the paraffins are not separated. This combined paraffin and C₅+ olefin stream can be fed to an olefin cracking unit and/or to an OTO conversion system.

FIG. 1 shows the C₄ stream 44 being fed to a hydrogenation unit 54. All or part of the C₄ stream may be at least partially hydrogenated with a source of hydrogen. The at least partially dehydrogenated C₄ stream can be recycled to the OTO conversion system via line 57 and line 17. When recycling to the OTO, the recycle C₄ stream can be a co-feed to the OTO reaction zone or it can be a feed to an optional catalytic olefin cracking zone downstream from the OTO reaction zone. Suitable catalysts and conditions are described herein, as well as in U.S. Pat. No. 6,809,227 and US 2004/0102667. Catalysts include those comprising zeolite molecular sieves such as MFI-type, e.g., ZSM-5, or MEL-type, e.g., ZSM-11, as well as Boralite-D and silicalite 2.

In one particular embodiment, the stream 44 comprises a small quantity of di-olefins, in particular butadiene. A small quantity of butadiene is for example, at least 0.01 wt % of butadiene in the stream, in particular at least 0.1 wt %, more in particular at least 0.5 wt. The stream comprising a small quantity of butadiene may be subjected to selective hydrogenation conditions in hydrogenation unit 54 to convert butadiene to butene, but preferably minimizing the hydrogenation of butene to butane. A suitable process for selective hydrogenation is described in U.S. Pat. No. 4,695,560. It is preferred for at least 90 wt % of the butadiene to be converted to butene and less than 10 wt %, preferably less than 5 wt % of the butene to be converted to butane. In another embodiment, the small quantity of butadiene may be left in the stream and recycled to the OTO conversion system. Other di-olefins, including C₅ di-olefins may also be present in the conversion effluent and may need to be selectively hydrogenated in like manner before recycling the C₅ olefin or paraffin streams.

The effluent from selective hydrogenation is a C₄ feedstock comprising butene, and butene is a desirable co-feed in OTO reactions, in particular in the MtP process or in a process in which a catalyst comprising an aluminosilicate or zeolite having one-dimensional 10-membered ring channels and an olefin co-feed is employed. The butene rich effluent can be recycled via line 57.

An optional cracking unit 80 may be used to convert lower paraffins, ethane and propane, to additional lower olefins which can then be fed to the workup section 60 via line 82.

### EXAMPLES

### Example 1

Two catalysts, comprising 40 wt % zeolite, 36 wt % kaolin and 24 wt % silica were tested to show their ability to convert isopentane to an olefinic product. To test the catalyst formulations for catalytic performance, the catalysts were pressed into tablets and the tablets were broken into pieces and sieved.

In the preparation of the first catalyst sample, ZSM-23 zeolite powder with a silica to alumina molar ratio (SAR) of 46, and ZSM-5 zeolite powder with a SAR of 80 were used in the ammonium form in the weight ratio 50:50. Prior to mixing the powders, the ZSM-5 zeolite powder was treated with phosphorus, resulting in a catalyst that has only one zeolite pre-treated with phosphorus. Phosphorus was deposited on a ZSM-5 zeolite powder with a silica-to-alumina ratio of 80 by means of impregnation with an acidic solution containing phosphoric acid to obtain a ZSM-5 treated zeolite powder containing 2.0 wt % P. The ZSM-5 powder was calcined at 550° C. Then, the powder mix was added to an aqueous solution and subsequently the slurry was milled. Next, kaolin clay and a silica sol were added and the resulting mixture was spray dried wherein the weight-based average particle size was between 70-90 μm. The spray dried catalysts were exposed to ion-exchange using an ammonium nitrate solution. Then, phosphorus was deposited on the catalyst by means of impregnation using acidic solutions containing phosphoric acid (H₃PO₄). The concentration of the solution was adjusted to impregnate 1.0 wt % of phosphorus on the catalyst. After impregnation the catalysts were dried at 140° C. and then calcined at 550° C. for 2 hours. The final formulated catalyst thus obtained is further referred to as catalyst 1.

Another formulated catalyst was prepared as described herein above for catalyst 1, with the exception that only ZSM-5 with a SAR of 80 was used and it was not treated with phosphorus prior to spray-drying. The concentration of the phosphorus impregnation solution was adjusted to impregnate 1.5 wt % of phosphorus on the catalyst formulation. The final formulated catalyst thus obtained is further referred to as catalyst 2.

The phosphorus loading on the final catalysts is given based on the weight percentage of the elemental phosphorus in any phosphor species, based on the total weight of the formulated catalyst.

Isopentane was reacted over the catalysts which were tested to determine their selectivity towards olefins, mainly ethylene and higher olefins. For the catalytic testing, a sieve fraction of 60-80 mesh was used. The reaction was performed using a quartz reactor tube of 1.8 mm internal diameter. The molecular sieve samples were heated in nitrogen to the reaction temperature and a mixture consisting of 3 vol % isopentane and, in some tests, 6 vol % methanol, balanced with N₂ was passed over the catalyst at atmospheric pressure (1 bar).

The Gas Hourly Space Velocity (GHSV) is determined by the total gas flow over the zeolite weight per unit time (ml gas)/(g zeolite·hr). The gas hourly space velocity used in the experiments was 19,000 (ml gas)/(g zeolite·hr). The effluent from the reactor was analyzed by gas chromatography (GC) to determine the product composition. The composition was calculated on a weight basis of all hydrocarbons analyzed. The composition was defined by the division of the mass of specific product by the sum of the masses of all products. The effluent from the reactor obtained at several reactor temperatures was analyzed. The results are shown in Table 1.

TABLE 1

| | | | Light | | | | | | |
|---|---|---|---|---|---|---|---|---|---|
| | Methanol | Temp | Ends | C₂ tot | C₃ tot | C₄ tot | C₅₊ tot | BTX | C5 sat/ |
| Cat. | (vol. %) | (° C.) | (wt %) | (wt %) | (wt %) | (wt %) | (wt %) | (wt %) | C5 total |
| 1 | 6 | 525 | 1.23 | 5.10 | 20.17 | 8.70 | 64.59 | 0.21 | 98.42 |
| 1 | 6 | 600 | 3.00 | 8.89 | 22.39 | 10.06 | 54.92 | 0.74 | 98.16 |
| 1 | 6 | 630 | 4.10 | 10.62 | 22.74 | 10.54 | 50.95 | 1.04 | 97.65 |
| 1 | 0 | 525 | 0.52 | 0.91 | 1.43 | 1.75 | 95.39 | 0.00 | 99.31 |
| 1 | 0 | 600 | 2.64 | 4.28 | 6.03 | 8.45 | 78.60 | 0.00 | 98.26 |
| 1 | 0 | 630 | 4.08 | 6.17 | 8.55 | 12.08 | 69.11 | 0.00 | 98.00 |

TABLE 2

| | | | Light | | | | | | | | |
|---|---|---|---|---|---|---|---|---|---|---|---|
| | Methanol | Temp | Ends | C₂ tot | C₃ tot | C₄ tot | C₅₊ tot | B | C5 sat/ | Ethane | Propane |
| Cat. | (vol. %) | (° C.) | (wt %) | (wt %) | (wt %) | (wt %) | (wt %) | (wt %) | C5 total | (wt %) | (wt %) |
| 2 | 6 | 525 | 1.44 | 6.27 | 20.64 | 8.78 | 62.44 | 0.43 | 98.48 | 0.46 | 0.35 |
| 2 | 6 | 600 | 4.16 | 11.83 | 23.93 | 10.60 | 47.76 | 1.72 | 97.86 | 1.47 | 0.24 |
| 2 | 6 | 630 | 5.73 | 14.13 | 24.55 | 11.02 | 42.47 | 2.10 | 97.49 | 1.85 | 0.30 |
| 2 | 0 | 525 | 1.05 | 1.93 | 2.95 | 3.45 | 90.62 | 0.00 | 99.17 | 0.54 | 0.01 |
| 2 | 0 | 600 | 4.56 | 7.74 | 11.00 | 13.58 | 63.13 | 0.00 | 97.84 | 1.39 | 0.23 |
| 2 | 0 | 630 | 6.16 | 9.78 | 13.18 | 16.73 | 54.08 | 0.06 | 97.39 | 1.37 | 0.23 |

As can be seen from the examples, the isopentane fed to the reaction is converted to C4 olefins and methane, ethylene and propylene, and hydrogen and C₅ olefins. This shows that a recycle of C₅ paraffins can be effective in producing additional olefins. The small amounts of propane produced indicate that the isopentane does not crack to propane and ethylene in significant amounts. The C₅₊ stream produced is predominantly C₅ saturates (see C₅ sat/C₅ tot ratio).

1. A process for the preparation of an olefin product, which process comprises the steps of:
a. converting an oxygenate feedstock in an oxygenate-to-olefins conversion system, comprising a reaction zone in which an oxygenate feedstock is contacted with an oxygenate conversion catalyst under oxygenate conversion conditions, to obtain a conversion effluent comprising olefins and paraffins;
b. separating at least a portion of the paraffins from the conversion effluent to form a paraffin stream; and
c. recycling at least a portion of the paraffin stream to step a).

2. A process as claimed in claim 1 wherein the oxygenate conversion catalyst comprises at least one zeolite selected from MFI, MEL, TON and MTT type zeolites.

3. A process as claimed in claim 1 wherein the oxygenate conversion catalyst comprises at least one zeolite selected from ZSM-5, ZSM-11, ZSM-22 and ZSM-23 zeolites.

4. A process as claimed in claim 1 wherein the oxygenate conversion catalyst comprises ZSM-5 that has been phosphorized.

5. A process as claimed in claim 1 wherein the oxygenate conversion conditions comprise a temperature in the range of from 600° C. to 660° C. and a pressure in the range of from 0.1 kPa to 5 MPa.

6. A process as claimed in claim 1 wherein the oxygenate conversion conditions comprise a temperature in the range of from 600° C. to 640° C. and a pressure in the range of from 100 kPa to 1.5 MPa.

7. A process as claimed in claim 1 wherein the oxygenate conversion conditions comprise a temperature in the range of from 620° C. to 640° C.

8. A process as claimed in claim 1 further comprising recycling at least a portion of the olefins to step a).

9. A process as claimed in 6 wherein the recycled olefins comprise olefins having from 4 to 6 carbon atoms.

10. A process as claimed in claim 1 wherein the oxygenate feedstock is selected from the group consisting of methanol, ethanol, tert-alkyl ethers and mixtures thereof.

11. A process as claimed in claim 1 further comprising passing the recycled paraffins to an olefin cracking unit where they are contacted with an olefin cracking catalyst under olefin cracking conditions to produce additional olefins that are fed along with any remaining paraffins to step (a).
